SEQUENCE LISTING (21 sequences)

SEQ ID NO: 1 - DNA, 1251 bp, Clostridium subterminale
  CDS (1)..(1248): Clostridial Lysine 2,3-aminomutase
atgataaatagaagatatgaattatttaaagatgttagcgatgcagactggaatgactggagatggcaagtaagaaacagaatagaaactgttgaagaactaaagaaatacataccattaacaaaagaagaagaagaaggagtagctcaatgtgtaaaatcattaagaatggctattactccatattatctatcattaatcgatcctaacgatcctaatgatccagtaagaaaacaagctattccaacagcattagagcttaacaaagctgctgcagatcttgaagacccattacatgaagatacagattcaccagtacctggattaactcacagatatccagatagagtattattattaataactgatatgtgctcaatgtactgcagacactgtacaagaagaagatttgcaggacaaagcgatgactctatgccaatggaaagaatagataaagctatagattatatcagaaatactcctcaagttagagacgtattattatcaggtggagacgctcttttagtatctgatgaaacattagaatacatcatagctaaattaagagaaataccacacgttgaaatagtaagaataggttcaagaactccagttgttcttccacaaagaataactccagaacttgtaaatatgcttaaaaaatatcatccagtatggttaaacactcactttaaccatccaaatgaaataacagaagaatcaactagagcttgtcaattacttgctgacgcaggagtacctctaggaaaccaatcagttttattaagaggagttaacgattgcgtacacgtaatgaaagaattagttaacaaattagtaaaaataagagtaagaccttactacatctatcaatgtgacttatcattaggacttgagcacttcagaactccagtttctaaaggtatcgaaatcattgaaggattaagaggacatacttcaggatactgcgtaccaacattcgttgttgacgctccaggtggtggtggaaaaacaccagttatgccaaactacgttatttcacaaagtcatgacaaagtaatattaagaaactttgaaggtgttataacaacttattcagaaccaataaactatactccaggatgcaactgtgatgtttgcactggcaagaaaaaagttcataaggttggagttgctggattattaaacggagaaggaatggctctagaaccagtaggattagagagaaataagagacacgttcaagaataa

SEQ ID NO: 2 - protein, 416 aa, Clostridium subterminale
  Clostridial Lysine 2,3-aminomutase
MINRRYELFKDVSDADWNDWRWQVRNRIETVEELKKYIPLTKEEEEGVAQCVKSLRMAITPYYLSLIDPNDPNDPVRKQAIPTALELNKAAADLEDPLHEDTDSPVPGLTHRYPDRVLLLITDMCSMYCRHCTRRRFAGQSDDSMPMERIDKAIDYIRNTPQVRDVLLSGGDALLVSDETLEYIIAKLREIPHVEIVRIGSRTPVVLPQRITPELVNMLKKYHPVWLNTHFNHPNEITEESTRACQLLADAGVPLGNQSVLLRGVNDCVHVMKELVNKLVKIRVRPYYIYQCDLSLGLEHFRTPVSKGIEIIEGLRGHTSGYCVPTFVVDAPGGGGKTPVMPNYVISQSHDKVILRNFEGVITTYSEPINYTPGCNCDVCTGKKKVHKVGVAGLLNGEGMALEPVGLERNKRHVQE

SEQ ID NO: 3 - DNA, 35 bp, Artificial Sequence
  Chemically synthesized primer for Escherichia coli
gagagagactcgagttctacgcgagtaccggtcag

SEQ ID NO: 4 - DNA, 35 bp, Artificial Sequence
  Chemically synthesized primer for Escherichia coli
caacagcaatgcatatgaataattaaaggttatgc

SEQ ID NO: 5 - DNA, 34 bp, Artificial Sequence
  Chemically synthesized primer for Corynebacterium glutamicum
gagagagactcgagtagctgccaattattccggg

SEQ ID NO: 6 - DNA, 40 bp, Artificial Sequence
  Chemically synthesized primer for Corynebacterium glutamicum
tacgaaaggattttttacccatggcgcatattgtaaccct

SEQ ID NO: 7 - DNA, 20 bp, Artificial Sequence
  Chemically synthesized primer for Clostridium subterminale
cagtctgcatcgctaacatc

SEQ ID NO: 8 - DNA, 20 bp, Artificial Sequence
  Chemically synthesized primer for Clostridium subterminale
ggctctagaaccagtaggat

SEQ ID NO: 9 - DNA, 35 bp, Artificial Sequence
  Chemically synthesized primer for Corynebacterium glutamicum
gagagagagctcgagaagctttttaatcgaggcgt

SEQ ID NO: 10 - DNA, 41 bp, Artificial Sequence
  Chemically synthesized primer for Corynebacterium glutamicum
ctctctctcacgcgtaagcttgagctgctgatatgtcaggc

SEQ ID NO: 11 - DNA, 20 bp, Artificial Sequence
  Chemically synthesized primer for Corynebacterium glutamicum
tcccgaaagtttatggtgaa

SEQ ID NO: 12 - DNA, 34 bp, Artificial Sequence; identical to SEQ ID NO: 5
  Chemically synthesized primer for Corynebacterium glutamicum

SEQ ID NO: 13 - DNA, 40 bp, Artificial Sequence
  Chemically synthesized primer for Corynebacterium glutamicum
acgaaaggattttttacccatgaacatcattgccattatg

SEQ ID NO: 14 - DNA, 35 bp, Artificial Sequence
  Chemically synthesized primer for Corynebacterium glutamicum
ctctctctcactagtgctcaatcacatattgccca

SEQ ID NO: 15 - DNA, 34 bp, Artificial Sequence
  Chemically synthesized primer for Corynebacterium glutamicum
gagagagactcgagccggaagcgatggcggcatc

SEQ ID NO: 16 - DNA, 40 bp, Artificial Sequence
  Chemically synthesized primer for Corynebacterium glutamicum
tacgaaaggattttttacccatgagttctgccaagaagat

SEQ ID NO: 17 - DNA, 17 bp, Artificial Sequence
  Chemically synthesized primer for Clostridium subterminale
cctaacacagaaatgtc

SEQ ID NO: 18 - DNA, 16 bp, Artificial Sequence
  Chemically synthesized primer for Clostridium subterminale
tcctttgtaatatcgc

SEQ ID NO: 19 - DNA, 40 bp, Artificial Sequence
  Chemically synthesized primer for Corynebacterium glutamicum
atcttcttggcagaactcatgggtaaaaaatcctttcgta

SEQ ID NO: 20 - DNA, 34 bp, Artificial Sequence
  Chemically synthesized primer for Corynebacterium glutamicum
gagagagatctagatagctgccaattattccggg

SEQ ID NO: 21 - DNA, 21 bp, Artificial Sequence
  Chemically synthesized primer for Corynebacterium glutamicum
gggtaaaaaatcctttcgtag

(19) United States
(12) Patent Application Publication
(10) Pub. No.: US 2009/0029425 A1
(43) Pub. Date: Jan. 29, 2009

(54) PROCESS FOR THE PRODUCTION OF BETA-LYSINE

(75) Inventors: Oskar Zelder, Speyer (DE); Weo Kyu Jeong, Hirschberg (DE); Corinna Klopprogge, Mannheim (DE); Andrea Herold, Ketsch (DE); Hartwig Schroder, Nussloch (DE)

Correspondence Address: CONNOLLY BOVE LODGE & HUTZ, LLP, P O BOX 2207, WILMINGTON, DE, 19899, US

(73) Assignee: BASF SE

(21) Appl. No.: 12/281,789

(22) PCT Filed: Mar. 7, 2007
(86) PCT No.: PCT/EP07/52138

§ 371 Date: Sep. 5, 2008

(30) Foreign Application Priority Data
Mar. 9, 2006 (EP) 06110914.6

(51) Int. Cl. C12P 13/08; C12P 13/00; C12P 17/10

(52) U.S. Cl. 435/115; 435/121; 435/128

(57) ABSTRACT

Process for the production of -lysine by constructing a recombinant microorganism which has a deregulated lysine 2,3-aminomutase gene and at least one deregulated gene selected from the group (i) which consists of aspartokinase, aspartatesemialdehyde dehydrogenase, dihydrodipicolinate synthase, dihydrodipicolinate reductase, tetrahydrodipicolinate succinylase, succinyl-amino-ketopimelate transaminase, succinyl-diamino-pimelate desuccinylase, diaminopimelate epimerase, diamino-pimelate dehydrogenase, arginyl-tRNA synthetase, diaminopimelate decarboxylase, pyruvate carboxylase, phosphoenolpyruvate carboxylase, glucose-6-phosphate dehydrogenase, transketolase, transaldolase, 6-phosphogluconolactonase, fructose 1,6-biphosphatase, homoserine dehydrogenase, phophoenolpyruvate carboxykinase, succinyl-CoA synthetase, methylmalonyl-CoA mutase, provided that if aspartokinase is deregulated as gene (i) at least a second gene (i) other than aspartokinase has to be deregulated, and cultivating said microorganism.

### FIELD OF THE INVENTION

The present invention relates to a process for the production of β-lysine (beta-lysine) More particularly, this invention relates to the use of recombinant microorganism comprising DNA molecules in a deregulated form which are essential to produce β-lysine.

### RELATED ART

Although less abundant than the corresponding α-amino acids, β-amino acids occur in nature in both free forms and in peptides. Cardillo and Tomasini, Chem. Soc. Rev. 25:77 (1996); Sewald, Amino Acids 11:397 (1996). Since β-amino acids are stronger bases and weaker acids than α-amino acid counterparts, peptides that contain a β-amino acid in place of an α-amino acid, have a different skeleton atom pattern, resulting in new properties

In the 1950's, L-β-lysine was identified in several strongly basic peptide antibiotics produced by Streptomyces. Antibiotics that yield L-β-lysine upon hydrolysis include viomycin, streptolin A, streptothricin, roseothricin and geomycin. Stadtman, Adv. Enzymol. Relat. Areas Molec. Biol. 38:413 (1973). β-Lysine is also a constituent of antibiotics produced by the fungi Nocardia, such as mycomycin, and β-lysine may be used to pre-pare other biologically active compounds. However, the chemical synthesis of β-lysine is time consuming, requires expensive starting materials, and results in a racemic mixture.

### SUMMARY OF THE INVENTION

In one aspect, the present invention provides a process for the production of β-lysine by constructing a recombinant microorganism which has a deregulated lysine-2,3-aminomutase and at least one deregulated gene selected from genes which are essential in the lysine biosynthetic pathway, and cultivating said microorganism.

In another aspect, the present invention provides a process for the production of β-amino-ε-caprolactam comprising a step as mentioned above for the production of β-lysine.

In another aspect, the present invention provides a process for the production of ε-caprolactam comprising a step as mentioned above for the production of β-lysine.

### DETAILED DESCRIPTION OF THE INVENTION

In the description that follows, a number of terms are utilized extensively. Definitions are herein provided to facilitate understanding of the invention.

The term β-lysine means L-β-lysine.

Promoter. A DNA sequence which directs the transcription of a structural gene to produce mRNA. Typically, a promoter is located in the 5′ region of a gene, proximal to the start codon of a structural gene. If a promoter is an inducible promoter, then the rate of transcription increases in response to an inducing agent. In contrast, the rate of transcription is not regulated by an inducing agent, if the promoter is a constitutive promoter.

Enhancer. A promoter element. An enhancer can increase the efficiency with which a particular gene is transcribed into mRNA irrespective of the distance or orientation of the enhancer relative to the start site of transcription.

Expression. Expression is the process by which a polypeptide is produced from a structural gene. The process involves transcription of the gene into mRNA and the translation of such mRNA into polypeptide(s).

Cloning vector. A DNA molecule, such as a plasmid, cosmid, phagemid, or bacteriophage, which has the capability of replicating autonomously in a host cell and which is used to transform cells for gene manipulation. Cloning vectors typically contain one or a small number of restriction endonuclease recognition sites at which foreign DNA sequences may be inserted in a determinable fashion without loss of an essential biological function of the vector, as well as a marker gene which is suitable for use in the identification and selection of cells transformed with the cloning vector. Marker genes typically include genes that provide tetracycline resistance or ampicillin resistance.

Expression vector. A DNA molecule comprising a cloned structural gene encoding a foreign protein which provides the expression of the foreign protein in a recombinant host. Typically, the expression of the cloned gene is placed under the control of (i.e., operably linked to) certain regulatory sequences such as promoter and enhancer sequences. Promoter sequences may be either constitutive or inducible.

Recombinant host. A recombinant host may be any prokaryotic or eukaryotic cell which contains either a cloning vector or expression vector. This term is also meant to include those prokaryotic or eukaryotic cells that have been genetically engineered to contain the cloned gene(s) in the chromosome or genome of the host cell. For examples of suitable hosts, see Sambrook et al., MOLECULAR CLONING: A LABORATORY MANUAL, Second Edition, Cold Spring Harbor Laboratory, Cold Spring Harbor, N.Y. (1989) [“Sambrook”].

As used herein, a substantially pure protein means that the desired purified protein is essentially free from contaminating cellular components, as evidenced by a single band following polyacrylamide-sodium dodecyl sulfate gel electrophoresis (SDS-PAGE). The term “substantially pure” is further meant to describe a molecule which is homogeneous by one or more purity or homogeneity characteristics used by those of skill in the art. For example, a substantially pure lysine 2,3-aminomutase will show constant and reproducible characteristics within standard experimental deviations for parameters such as the following: molecular weight, chromatographic migration, amino acid composition, amino acid sequence, blocked or unblocked N-terminus, HPLC elution profile, biological activity, and other such parameters. The term, however, is not meant to exclude artificial or synthetic mixtures of lysine 2,3-aminomutase with other compounds. In addition, the term is not meant to exclude lysine 2,3-aminomutase fusion proteins isolated from a recombinant host.

In a first aspect, the present invention provides a process for the production of P-lysine by constructing a recombinant microorganism which has a deregulated lysine-2,3-aminomutase and at least one deregulated gene selected from the group (i) which consists of aspartokinase, aspartatesemialdehyde dehydrogenase, dihydrodipicolinate synthase, dihydrodipicolinate reductase, tetrahydrodipicolinate succinylase, succinyl-amino-ketopimelate transaminase, succinyl-diamino-pimelate desuccinylase, diamino-pimelate epimerase, diaminopimelate dehydrogenase, arginyl-tRNA synthetase, diamino-pimelate decarboxylase, pyruvate carboxylase, phosphoenolpyruvate carboxylase, glucose-6-phosphate dehydrogenase, transketolase, transaldolase, 6-phosphogluconolactonase, fructose 1,6-biphosphatase, homoserine dehydrogenase, phophoenol pyruvate carboxykinase, succi nyl-CoA synthetase, methylmalonyl-CoA mutase, provided that if aspartokinase is deregulated as gene (i) at least a second gene (i) other than aspartokinase has to be deregulated, and cultivating said microorganism.

The methodologies of the present invention feature recombinant microorganisms, preferably including vectors or genes (e.g., wild-type and/or mutated genes) as described herein and/or cultured in a manner which results in the production of P-lysine.

The term “recombinant” microorganism includes a microorganism (e.g., bacteria, yeast cell, fungal cell, etc.) which has been genetically altered, modified or engineered (e.g., genetically engineered) such that it exhibits an altered, modified or different genotype and/or phenotype (e.g., when the genetic modification affects coding nucleic acid sequences of the microorganism) as compared to the naturally-occurring microorganism from which it was derived.

The term “deregulated” includes expression of a gene product (e.g., lysine-2,3-aminomutase) at a level lower or higher than that expressed prior to manipulation of the microorganism or in a comparable microorganism which has not been manipulated. In one embodiment, the microorganism can be genetically manipulated (e.g., genetically engineered) to express a level of gene product at a lesser or higher level than that expressed prior to manipulation of the microorganism or in a comparable microorganism which has not been manipulated. Genetic manipulation can include, but is not limited to, altering or modifying regulatory sequences or sites associated with expression of a particular gene (e.g., by removing strong promoters, inducible promoters or multiple promoters), modifying the chromosomal location of a particular gene, altering nucleic acid sequences adjacent to a particular gene such as a ribosome binding site or transcription terminator, decreasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene routine in the art (including but not limited to use of antisense nucleic acid molecules, or other methods to knock-out or block expression of the target protein).

The term “deregulated lysine-2,3-aminomutase” also means that a lysine-2,3-aminomutase activity is introduced into a microorganism

where a lysine-2,3-aminomutase activity has not been observed before, e.g. by introducing a heterologous lysine-2,3-aminomutase gene in one or more copies into the microorganism preferably by means of genetic engineering.

Lysine 2,3-aminomutase catalyzes the reversible isomerization of L-lysine into β-lysine. The enzyme isolated from Clostridium subterminale strain SB4 is a hexameric protein of apparently identical subunits, which has a molecular weight of 285,000, as determined from diffusion and sedimentation coefficients. Chirpich et al., J. Biol. Chem. 245:1778 (1970); Aberhart et al., J. Am. Chem. Soc. 105:5461 (1983); Chang et al., Biochemistry 35:11081 (1996). The clostridial enzyme contains iron-sulfur clusters, cobalt and zinc, and pyridoxal 5′-phosphate, and it is activated by Sadenosylmethionine. Unlike typical adenosylcobalamin-dependent aminomutases, the clostridial enzyme does not contain or require any species of vitamin B₁₂ coenzyme.

The nucleotide and predicted amino acid sequences of clostridial lysine 2,3-aminomutase (SEQ ID NOs:1 and 2) are disclosed in U.S. Pat. No. 6,248,874B1.

DNA molecules encoding the clostridial lysine 2,3-aminomutase gene can be obtained by screening cDNA or genomic libraries with polynucleotide probes having nucleotide sequences based upon SEQ ID NO:1. For example, a suitable library can be prepared by obtaining genomic DNA from Clostridium subterminale strain SB4 (ATCC No. 29748) and constructing a library according to standard methods. See, for example, Ausubel et al. (eds.), SHORT PROTOCOLS IN MOLECULAR BIOLOGY, 3rd Edition, pages 2-1 to 2-13 and 5-1 to 5-6 (John Wiley & Sons, Inc. 1995).

Alternatively, the clostridial lysine 2,3-aminomutase gene can be obtained by synthesizing DNA molecules using mutually priming long oligonucleotides. See, for example, Ausubel et al., (eds.), CURRENT PROTOCOLS IN MOLECULAR BIOLOGY, pages 8.2.8 to 8.2.13 (1990) [“Ausubel”]. Also, see Wosnick et al., Gene 60:115 (1987); and Ausubel et al. (eds.), SHORT PROTOCOLS IN MOLECULAR BIOLOGY, 3rd Edition, pages 8-8 to 8-9 (John Wiley & Sons, Inc. 1995). Established techniques using the polymerase chain reaction provide the ability to synthesize DNA molecules at least 2 kilobases in length. Adang et al., Plant Molec. Biol. 21:1131 (1993); Bambot et al., PCR Methods and Applications 2:266 (1993); Dillon et al., “Use of the Polymerase Chain Reaction for the Rapid Construction of Synthetic Genes,” in METHODS IN MOLECULAR BIOLOGY, Vol. 15: PCR PROTOCOLS: CURRENT METHODS AND APPLICATIONS, White (ed.), pages 263-268, (Humana Press, Inc. 1993); Holowachuk et al., PCR Methods Appl. 4:299 (1995).

Variants of clostridial lysine 2,3-aminomutase can be produced that contain conservative amino acid changes, compared with the parent enzyme. That is, variants can be obtained that contain one or more amino acid substitutions of SEQ ID NO:2, in which an alkyl amino acid is substituted for an alkyl amino acid in the clostridial lysine 2,3-aminomutase amino acid sequence, an aromatic amino acid is substituted for an aromatic amino acid in the clostridial lysine 2,3-aminomutase amino acid sequence, a sulfur-containing amino acid is substituted for a sulfur-containing amino acid in the clostridial lysine 2,3-aminomutase amino acid sequence, a hydroxy-containing amino acid is substituted for a hydroxy-containing amino acid in the clostridial lysine 2,3-aminomutase amino acid sequence, an acidic amino acid is substituted for an acidic amino acid in the clostridial lysine 2,3-aminomutase amino acid sequence, a basic amino acid is substituted for a basic amino acid in the clostridial lysine 2,3-aminomutase amino acid sequence, or a dibasic monocarboxylic amino acid is substituted for a dibasic monocarboxylic amino acid in the clostridial lysine 2,3-aminomutase amino acid sequence.

Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) cysteine and methionine, (4) serine and threonine, (5) aspartate and glutamate, (6) glutamine and asparagine, and (7) lysine, arginine and histidine.

Conservative amino acid changes in the clostridial lysine 2,3-aminomutase can be introduced by substituting nucleotides for the nucleotides recited in SEQ ID NO:1. Such “conservative amino acid” variants can be obtained, for example, by oligonucleotidedirected mutagenesis, linker-scanning mutagenesis, mutagenesis using the polymerase chain reaction, and the like. Ausubel et al., supra, at pages 8.0.3-8.5.9; Ausubel et al. (eds.), SHORT PROTOCOLS IN MOLECULAR BIOLOGY, 3rd Edition, pages 8-10 to 8-22 (John Wiley & Sons, Inc. 1995). Also see generally, McPherson (ed.), DIRECTED MUTAGENESIS: A PRACTICAL APPROACH, IRL Press (1991). The ability of such variants to convert L-lysine to L-β-lysine can be determined using a standard enzyme activity assay, such as the assay described herein.

Lysine-2,3-aminomutases from other sources than from Clostridium subterminale, e.g. from Bacillus subtilis or from Escherichia coli have been disclosed in U.S. Pat. No. 6,248,874B1. The parts of this US patent dealing with the isolation, SEQ ID NOs and expression of lysine-2,3-aminomutases are herewith incorporated by reference expressly.

Preferred lysine-2,3-aminomutases according to the invention are the lysine-2,3-aminomutase from Clostridium subterminale, Bacillus subtilis and Escherichia coli and their equivalent genes, which have up to 80%, preferably 90%, most preferred 95% and 98% sequence identity (based on amino acid sequence) with the corresponding “original” gene product and have still the biological activity of lysine 2,3-aminomutase. These equivalent genes can be easily be constructed by introducing nucleotide substitutions, deletions or insertions by methods known in the art.

Another preferred embodiment of the invention is the lysine-2,3-aminomutase from Clostridium subterminale (SEQ ID NO:2 of U.S. Pat. No. 6,248,874B1) which is retranslated into DNA by applying the codon usage of Corynebacterium glutamicum. This lysine-2,3-aminomutase polynucleotide sequence is useful for expression of lysine 2,3-aminomutase in microorganism of the genus Corynebacterium, especially C. glutamicum.

In addition to the deregulated lysine 2,3-aminomutase gene the microorganism according to the invention must have at least one deregulated gene selected from the group (i). The group (i) is a group of genes which play a key role in the biosynthesis of lysine and consists of the genes of aspartokinase, aspartatesemialdehyde dehydrogenase, dihydrodipicolinate synthase, dihydrodipicolinate reductase, tetrahydrodipicolinate succinylase, succinyl-amino-ketopimelate transaminase, succinyl-diamino-pimelate desuccinylase, diaminopimelate epimerase, diaminopimelate dehydrogenase, arginyl-tRNA synthetase, diaminopimelate decarboxylase, pyruvate carboxylase, phosphoenolpyruvate carboxylase, glucose-6-phosphate dehydrogenase, transketolase, transaldolase, 6-phosphogluconolactonase, fructose 1,6-biphosphatase, homoserine dehydrogenase, phophoenolpyruvate carboxykinase, succinyl-CoA synthetase, methylmalonyl-CoA mutase.

At least one gene of the group (i) has to be deregulated according to the inventive process. Preferably more than one gene of group (i), e.g. two, three, four, five, six, seven, eight, nine, ten genes are deregulated in the microorganism according to the invention.

The genes and gene products of group (i) are known in the art. EP 1108790 discloses mutations in the genes of homoserinedehydrogenase and pyruvatecarboxylase which have a beneficial effect on the productivity of recombinant corynebacteria in the production of lysine. WO 00/63388 discloses mutations in the gene of aspartokinase which have a beneficial effect on the productivity of recombinant corynebacteria in the production of lysine. EP 1108790 and WO 00/63388 are incorporated by reference with respect to the mutations in these genes described above.

In the table below for every gene/gene product possible ways of deregulation of the respective gene are mentioned. The literature and documents cited in the row “Deregulation” of the table are herewith incorporated by reference with respect to gene deregulation. The ways mentioned in the table are preferred embodiments of a deregulation of the respective gene.

TABLE 1

| Enzyme (gene product) | Gene | Deregulation |
|---|---|---|
| Aspartokinase | ask | Releasing feedback inhibition by |
| | | point mutation (Eggeling et al., |
| | | (eds.), Handbook of Corynebacterium |
| | | glutamicum, pages 20.2.2 |
| | | (CRC press, 2005)) and amplification) |
| Aspartatesemialdehyde dehydrogenase | asd | Amplification |
| Dihydrodipicolinate synthase | dapA | Amplification |
| Dihydrodipicolinate reductase | dapB | Amplification |
| Tetrahydrodipicolinate succinylase | dapD | Amplification |
| Succinyl-amino-ketopimelate | dapC | Amplification |
| transaminase | | |
| Succinyl-diamino-pimelate desuccinylase | dapE | Amplification |
| Diaminopimelate dehydrogenase | ddh | Amplification |
| Diaminopimelate epimerase | dapF | Amplification |
| Arginyl-tRNA synthetase | argS | Amplification |
| Diaminopimelate decarboxylase | lysA | Amplification |
| Pyruvate carboxylase | pycA | Releasing feedback inhibition by |
| | | point mutation (EP1108790) and |
| | | amplification |
| Phosphoenolpyruvate carboxylase | ppc | Amplification |
| Glucose-6-phosphate dehydrogenase | zwf | Releasing feedback inhibition by |
| | | point mutation (US2003/0175911) |
| | | and amplification |
| Transketolase | tkt | Amplification |
| Transaldolase | tal | Amplification |
| 6-Phosphogluconolactonase | pgl | Amplification |
| Fructose 1,6-biphosphatase | fbp | Amplification |
| Homoserine dehydrogenase | hom | Attenuating by point mutation |
| | | (EP1108790) |
| Phophoenolpyruvate carboxykinase | pck | Knock-out or silencing by mutation |
| | | or others |
| Succinyl-CoA synthetase | sucC | Attenuating by point mutation (WO |
| | | 05/58945) |
| Methylmalonyl-CoA mutase | | Attenuating by point mutation (WO |
| | | 05/58945) |

A preferred way of deregulation of the genes of aspartokinase, aspartatesemialdehyde dehydrogenase, dihydrodipicolinate synthase, dihydrodipicolinate reductase, tetrahydrodipicolinate succinylase, succinyl-amino-ketopimelate transaminase, succinyl-diamino-pimelate desuccinylase, diaminopimelate epimerase, diaminopimelate dehydrogenase, arginyl-tRNA synthetase, diaminopimelate decarboxylase, pyruvate carboxylase, phosphoenolpyruvate carboxylase, glucose-6-phosphate dehydrogenase, transketolase, transaldolase, 6-phosphogluconolactonase, fructose 1,6-biphosphatase is an “up”—mutation which increases the gene activity e.g. by gene amplification using strong expression signals and/or point mutations which enhance the enzymatic activity.

A preferred way of deregulation of the genes of homoserine dehydrogenase, phophoenolpyruvate carboxykinase, succinyl-CoA synthetase, methylmalonyl-CoA mutase is a “down”—mutation which decreases the gene activity e.g. by gene deletion or disruption, using weak expression signals and/or point mutations which destroy or decrease the enzymatic activity.

If aspartokinase is deregulated as a member of gene (i) group at least a second gene (i) member—other than aspartokinase—has to be deregulated also.

To express the deregulated genes according to the invention, the DNA sequence encoding the enzyme must be operably linked to regulatory sequences that control transcriptional expression in an expression vector and then, introduced into either a prokaryotic or eukaryotic host cell. In addition to transcriptional regulatory sequences, such as promoters and enhancers, expression vectors can include translational regulatory sequences and a marker gene which is suitable for selection of cells that carry the expression vector.

Suitable promoters for expression in a prokaryotic host can be repressible, constitutive, or inducible. Suitable promoters are well-known to those of skill in the art and include promoters capable of recognizing the T4, T3, Sp6 and T7 polymerases, the P_{R} and P_{L} promoters of bacteriophage lambda, the trp, recA, heat shock, lacUV5, tac, Ipp-lackpr, phoA, gal, trc and lacZ promoters of E. coli, the α-amylase and the σ²⁸-specific promoters of B. subtilis, the promoters of the bacteriophages of Bacillus, Streptomyces promoters, the int promoter of bacteriophage lambda, the bla promoter of the β-lactamase gene of pBR322, and the CAT promoter of the chloramphenicol acetyl transferase gene. Prokaryotic promoters are reviewed by Glick, J. Ind. Microbiol. 1:277 (1987); Watson et al., MOLECULAR BIOLOGY OF THE GENE, 4th Ed., Benjamin Cummins (1987); Ausubel et al., supra, and Sambrook et al., supra.

A preferred promoter for the expression of the lysine-2,3-aminomutase is the sodA promoter of C. glutamicum. In order to improve expression a terminator, e.g. the groEL terminator of C. glutamicum can be inserted downstream of the lysine-2,3-aminomutase gene.

Methods for expressing proteins in prokaryotic hosts are well-known to those of skill in the art. See, for example, Williams et al., “Expression of foreign proteins in E. coli using plasmid vectors and purification of specific polyclonal antibodies,” in DNA CLONING 2: EXPRESSION SYSTEMS, 2nd Edition, Glover et al. (eds.), pages 15-58 (Oxford University Press 1995). Also see, Ward et al., “Genetic Manipulation and Expression of Antibodies,” in MONOCLONAL ANTIBODIES: PRINCIPLES AND APPLICATIONS, pages 137-185 (Wiley-Liss, Inc. 1995); and Georgiou, “Expression of Proteins in Bacteria,” in PROTEIN ENGINEERING: PRINCIPLES AND PRACTICE, Cleland et al. (eds.), pages 101-127 (John Wiley & Sons, Inc. 1996).

An expression vector can be introduced into bacterial host cells using a variety of techniques including calcium chloride transformation, electroporation, and the like. See, for example, Ausubel et al. (eds.), SHORT PROTOCOLS IN MOLECULAR BIOLOGY, 3rd Edition, pages 1-1 to 1-24 (John Wiley & Sons, Inc. 1995).

An important aspect of the present invention involves cultivating or culturing the recombinant microorganisms described herein, such that a desired compound β-lysine is produced. The term “cultivating” includes maintaining and/or growing a living microorganism of the present invention (e.g., maintaining and/or growing a culture or strain). In one embodiment, a microorganism of the invention is cultured in liquid media. In another embodiment, a microorganism of the invention is cultured in solid media or semi-solid media. In a preferred embodiment, a microorganism of the invention is cultured in media (e.g., a sterile, liquid media) comprising nutrients essential or beneficial to the maintenance and/or growth of the microorganism.

Carbon sources which may be used include sugars and carbohydrates, such as for example glucose, sucrose, lactose, fructose, maltose, molasses, starch and cellulose, oils and fats, such as for example soy oil, sunflower oil, peanut oil and coconut oil, fatty acids, such as for example palmitic acid, stearic acid and linoleic acid, alcohols, such as for example glycerol and ethanol, and organic acids, such as for example acetic acid. In a preferred embodiment, glucose, fructose or sucrose are used as carbon sources. These substances may be used individually or as a mixture.

Nitrogen sources which may be used comprise organic compounds containing nitrogen, such as peptones, yeast extract, meat extract, malt extract, corn steep liquor, soya flour and urea or inorganic compounds, such as ammonium sulfate, ammonium chloride, ammonium phosphate, ammonium carbonate and ammonium nitrate. The nitrogen sources may be used individually or as a mixture. Phosphorus sources which may be used are phosphoric acid, potassium dihydrogen phosphate or dipotassium hydrogen phosphate or the corresponding salts containing sodium. The culture medium must furthermore contain metal salts, such as for example magnesium sulfate or iron sulfate, which are necessary for growth. Finally, essential growth-promoting sub-stances such as amino acids and vitamins may also be used in addition to the above-stated substances. Suitable precursors may furthermore be added to the culture medium. The stated feed substances may be added to the culture as a single batch or be fed appropriately during cultivation.

Preferably, microorganisms of the present invention are cultured under controlled pH. The term “controlled pH” includes any pH which results in production of the desired fine chemical, e.g., β-lysine. In one embodiment, microorganisms are cultured at a pH of about 7. In another embodiment, microorganisms are cultured at a pH of between 6.0 and 8.5. The desired pH may be maintained by any number of methods known to those skilled in the art. For example, basic compounds such as sodium hydroxide, potassium hydroxide, ammonia, or ammonia water, or acidic compounds, such as phosphoric acid or sulfuric acid, are used to appropriately control the pH of the culture.

Also preferably, microorganisms of the present invention are cultured under controlled aeration. The term “controlled aeration” includes sufficient aeration (e.g., oxygen) to result in production of the desired fine chemical, e.g., β-lysine. In one embodiment, aeration is controlled by regulating oxygen levels in the culture, for example, by regulating the amount of oxygen dissolved in culture media. Preferably, aeration of the culture is controlled by agitating the culture. Agitation may be provided by a propeller or similar mechanical agitation equipment, by revolving or shaking the growth vessel (e.g., fermentor) or by various pumping equipment. Aeration may be further controlled by the passage of sterile air or oxygen through the medium (e.g., through the fermentation mixture). Also preferably, microorganisms of the present invention are cultured without excess foaming (e.g., via addition of antifoaming agents such as fatty acid polyglycol esters).

Moreover, microorganisms of the present invention can be cultured under controlled temperatures. The term “controlled temperature” includes any temperature which results in production of the desired fine chemical, e.g., P-lysine. In one embodiment, controlled temperatures include temperatures between 15° C. and 95° C. In another embodiment, controlled temperatures include temperatures between 15° C. and 70° C. Preferred temperatures are between 20° C. and 55° C., more preferably between 30° C. and 45° C. or between 30° C. and 50° C.

Microorganisms can be cultured (e.g., maintained and/or grown) in liquid media and preferably are cultured, either continuously or intermittently, by conventional culturing methods such as standing culture, test tube culture, shaking culture (e.g., rotary shaking culture, shake flask culture, etc.), aeration spinner culture, or fermentation. In a preferred embodiment, the microorganisms are cultured in shake flasks. In a more preferred embodiment, the microorganisms are cultured in a fermentor (e.g., a fermentation process). Fermentation processes of the present invention include, but are not limited to, batch, fed-batch and continuous methods of fermentation. The phrase “batch process” or “batch fermentation” refers to a closed system in which the composition of media, nutrients, supplemental additives and the like is set at the beginning of the fermentation and not subject to alteration during the fermentation, however, attempts may be made to control such factors as pH and oxygen concentration to pre-vent excess media acidification and/or microorganism death. The phrase “fed-batch process” or “fed-batch” fermentation refers to a batch fermentation with the exception that one or more substrates or supplements are added (e.g., added in increments or continuously) as the fermentation progresses. The phrase “continuous process” or “continuous fermentation” refers to a system in which a defined fermentation medium is added continuously to a fermentor and an equal amount of used or “conditioned” medium is simultaneously removed, preferably for recovery of the desired P-lysine. A variety of such processes have been developed and are well-known in the art.

The methodology of the present invention can further include a step of recovering β-lysine. The term “recovering” β-lysine includes extracting, harvesting, isolating or purifying the compound from culture media. Recovering the compound can be performed according to any conventional isolation or purification methodology known in the art including, but not limited to, treatment with a conventional resin (e.g., anion or cation exchange resin, non-ionic adsorption resin, etc.), treatment with a conventional adsorbent (e.g., activated charcoal, silicic acid, silica gel, cellulose, alumina, etc.), alteration of pH, solvent extraction (e.g., with a conventional solvent such as an alcohol, ethyl acetate, hexane and the like), distillation, dialysis, filtration, concentration, crystallization, recrystallization, pH adjustment, lyophilization and the like. For example β-lysine can be recovered from culture media by first removing the microorganisms. The broth removed biomass is then passed through or over a cation exchange resin to remove unwanted cations and then through or over an anion exchange resin to remove unwanted inorganic anions and organic acids having stronger acidities than β-lysine.

In another aspect, the present invention provides a process for the production of β-amino-ε-caprolactam comprising a step as mentioned above for the production of β-lysine. β-Lysine undergoes an intramolecular cyclization resulting in β-amino-ε-caprolactam. This cyclization reaction can be performed either directly before the isolation and/or purification of the β-lysine or with the isolated β-lysine.

In another aspect, the present invention provides a process for the production of ε-caprolactam comprising a step as mentioned above for the production of β-lysine. As described above β-lysine can make an intramolecular cyclization resulting in β-amino-ε-caprolactam, which can be deaminated selectively in order to get ε-caprolactam. This deamination process is known in the art.

Another aspect of the present invention is the a process for the production of acid comprising a step as mentioned above for the production of β-lysine and subsequent deamination of the β-aminofunction of β-lysine. The resulting ε-aminocaproic acid can be transformed either to ε-caprolactam or directly—without cyclization to the lactam—to a polyamide by known polymerization techniques.

ε-Caprolactam is a very important monomer for the production of polyamides, especially PA6.

### EXAMPLES

### 1. Cloning of C. subterminale Lysine 2,3-aminomutase Gene

With conserved regions of the up- and downstream of the lysine 2,3-aminomutase gene in Fusobacterium nucleatum and Thermoanaerobacter tengcongensis, a set of oligonucleotide primers was designed to isolate C. subterminale lysine 2,3-aminomutase gene (kamA). PCR primers, WKJ90/WKJ65 and WKJ68/WKJ93, were used with the chromosome of C. subterminale as a template to amplify a DNA fragment of the up- and down-stream region including N- and C-terminal sequence of the kamA gene, respectively. The sequence analysis with amplified DNA fragments was carried out following purification and resulted in products containing start and end sequence of the kamA structural region. Based on determined the up- and downstream sequence PCR primers, WKJ105/WKJ106, were synthesized and used to isolate full sequence of the C. subterminale kamA gene. The amplified PCR fragment was purified, digested with restriction enzymes Xho I and Mlu I and ligated to the pClik5aMCS vector digested with same restriction enzymes (pClik5aMCS kamA).

### 2. Cloning of C. subterminale Synthetic Lysine 2,3-aminomutase Gene

The codon usage for the C. subterminale kamA gene is quite different with that for the C. glutamicum and this may lead to decrease of gene expression in C. glutamicum lysine producing strain. To enhance gene expression in C. glutamicum, synthetic kamA gene, which was adapted to C. glutamicum codon usage and had C. glutamicum sodA promoter (Psod) and groEL terminator instead of its own, was created. The synthetic kamA gene showed 72% of similarity on the nucleotide sequence compared with original one. Synthetic kamA gene had been cloned into the pClik5aMCS vector (pClik5aMCS syn_kamA).

### 3. Cloning of B. subtilis Lysine 2,3-aminomutase Gene

The DNA fragment containing B. subtilis lysine 2,3-aminomutase gene (yodo) was amplified from chromosomal DNA using PCR primers, WKJ71/WKJ72. The amplified DNA fragment was purified, digested with Xho I and Mlu I, and inserted between Xho I and Mlu I cleavage sites of the pClik5aMCS vector (pClik5aMCS yodo).

To increase expression of the gene, the C. glutamicum sodA promoter was substituted in front of coding region of yodO gene. The DNA fragments containing the sodA promoter and upstream region of the yodO gene were amplified from each chromosomal DNA using PCR primers WKJ75/WKJ78 and WKJ73/WKJ76, respectively and used as a template for fusion PCR with primers WKJ73/WKJ78 to make yodO upstream-Psod product. Subsequently, the Psod-controlled yodO gene was created by fusion PCR with WKJ73/WKJ74 as primers and yodO upstream-Psod and yodO coding region which was amplified with primer WKJ77/WKJ74 as templates. The PCR product was purified, digested with Xho I and Mlu I, and inserted to the pClik5aMCS vector (pClik5aMCS Psod yodo).

### 4. Cloning of E. coli Lysine 2,3-aminomutase Gene

PCR primers WKJ29/WKJ30 were used with the chromosome of E. coli as a template to amplify the lysine 2,3-aminomutase gene (yjeK). The amplified PCR fragment was purified, digested with restriction enzymes Xho I and Nde I and ligated to the pClik5aMCS vector digested with same restriction enzymes (pClik5aMCS yjek).

To increase expression of the gene, C. glutamicum sodA promoter was substituted in front of start codon of the yjek gene. The DNA fragments containing the sodA promoter and coding region of the yjek gene including the downstream region were amplified from each chromosomal DNA using PCR primers WKJ31/OLD47 and WKJ32/WKJ30, respectively, and used as a template for fusion PCR with primers WKJ31/WKJ30 to make Psodyjek gene. The PCR fragment was purified, digested with Xho I and Nde I, and inserted into Xho I-Nde I cleavage sites of the pClik5aMCS vector (pClik5aMCS Psod yjek).

Oligonucleotide primers used:

| | |
|---|---|
| WKJ29 | gagagagactcgagttctacgcgagtaccggtcag |
| WKJ30 | caacagcaatgcatatgaataattaaaggttatgc |
| WKJ31 | gagagagactcgagtagctgccaattattccggg |
| WKJ32 | tacgaaaggattttttacccatggcgcatattgtaaccct |
| WKJ65 | cagtctgcatcgctaacatc |
| WKJ68 | ggctctagaaccagtaggat |
| WKJ71 | gagagagagctcgagaagctttttaatcgaggcgt |
| WKJ72 | ctctctctcacgcgtaagcttgagctgctgatatgtcaggc |
| WKJ73 | tcccgaaagtttatggtgaa |
| WKJ74 | gagagagactcgagtagctgccaattattccggg |
| WKJ75 | acgaaaggattttttacccatgaacatcattgccattatg |
| WKJ76 | ctctctctcactagtgctcaatcacatattgccca |
| WKJ77 | gagagagactcgagccggaagcgatggcggcatc |
| WKJ78 | tacgaaaggattttttacccatgagttctgccaagaagat |
| WKJ90 | cctaacacagaaatgtc |
| WKJ93 | tcctttgtaatatcgc |
| WKJ105 | atcttcttggcagaactcatgggtaaaaaatcctttcgta |
| WKJ106 | gagagagatctagatagctgccaattattccggg |
| OLD47 | gggtaaaaaatcctttcgtag |

TABLE 2
Plasmids used

| plasmid | Characteristics |
|---|---|
| pClik5aMCS | E. coli/C. glutamicum shuttle vector, Km^{r} |
| pClik5aMCS kamA | pClik5aMCS carrying C. subterminale lysine 2,3- |
| | aminomutase gene (kamA) |
| pClik5aMCS syn_kamA | pClik5aMCS carrying C. subterminale synthetic kamA consisting |
| | of sodA promoter, kamA gene adapted to C. glutamicum |
| | codonusage and groEL terminator |
| pClik5aMCS yodO | pClik5aMCS carrying B. subtilis lysine 2,3-aminomutase |
| | gene (yodO) |
| pClik5aMCS Psod yodO | pClik5aMCS carrying B. subtilis yodO fused with C. glutamicum |
| | sodA promoter |
| pClik5aMCS yjeK | pClik5aMCS carrying E. coli lysine 2,3-aminomutase gene |
| | (yjeK) |
| pClik5aMCS Psod yjeK | pClik5aMCS carrying E. coli yjeK fused with C. glutamicum |
| | sodA promoter |

### 5. Construction of β-Lysine Production Strain of C. glutamicum

To construct recombinant β-lysine production strain, a lysine producer LU11271, which was constructed from C. glutamicum wild type strain ATCC13032 by incorporation of a point mutation T311I into aspartokinase gene, duplication of diaminopimelate dehydrogenase gene and disruption of phosphoenolpyruvate carboxykinase gene, was transformed with the recombinant plasmids having the lysine 2,3-aminomuatse genes.

### 6. β-Lysine Production in Shaking Flask Culture

Shaking flask experiments were performed on the recombinant strains to test β-lysine production. The same culture medium and conditions as lysine production were employed as described in WO2005059139. For the control, host strain and recombinant strain having pClik5aMCS were tested in parallel. The strains were precultured on CM agar overnight at 30° C. Cultured cells were harvested in a microtube containing 1.5 ml of 0.9% NaCl and cell density was determined by the absorbance at 610 nm following vortex. For the main culture, suspended cells were inoculated to reach 1.5 of initial OD into 10 ml of the production medium contained in an autoclaved 100 ml of Erlenmeyer flask having 0.5 g of CaCO₃. Main culture was performed on a rotary shaker (Infors AJ118, Bottmingen, Switzerland) with 200 rpm for 48-78 hours at 30° C. For cell growth measurement, 0.1 ml of culture broth was mixed with 0.9 ml of 1 N HCl to eliminate CaCO₃, and the absorbance at 610 nm was measured following appropriate dilution. The concentration of β-lysine, lysine and residual sugar including glucose, fructose and sucrose were measured by HPLC method (Agilent 1100 Series LC system).

As shown in tables below, an accumulation of β-lysine was observed in the broth cultured with recombinant strain containing C. subterminale synthetic kamA gene compared to the control strains. This indicates that the clostridial synthetic kamA gene functions in C. glutamicum. In addition, expression of the synthetic kamA gene was confirmed by SDS-PAGE.

TABLE 3
Shaking flask culture with C. clostridium kamA amplified strains

| | β- | |
|---|---|---|
| | Lysine(g/l) | OD610 nm |
| LU11271 | 0 | 46.9 |
| LU11271/pClik5aMCS | 0 | 47.8 |
| LU11271/pClik5aMCS syn_kamA | 0.2 | 44.3 |

1. (canceled)

2. (canceled)

3. (canceled)

4. (canceled)

5. (canceled)

6. (canceled)

7. (canceled)

8. (canceled)

9. (canceled)

10. A process for the production of Compound A, wherein Compound A is β-lysine, β-amino-ε-caprolactam, ε-caprolactam or ε-aminocaproic acid, and wherein the process comprises constructing a recombinant microorganism comprising a deregulated lysine 2,3-aminomutase gene and at least one deregulated gene selected from the group (i) consisting of genes encoding aspartokinase, aspartate semialdehyde dehydrogenase, dihydrodipicolinate synthase, dihydrodipicolinate reductase, tetrahydrodipicolinate succinylase, succinyl-amino-ketopimelate transaminase, succinyl-diamino-pimelate desuccinylase, diaminopimelate epimerase, diaminopimelate dehydrogenase, arginyl-tRNA synthetase, diaminopimelate decarboxylase, pyruvate carboxylase, phosphoenolpyruvate carboxylase, glucose-6-phosphate dehydrogenase, transketolase, transaldolase, 6-phosphogluconolactonase, fructose 1,6-biphosphatase, homoserine dehydrogenase, phophoenolpyruvate carboxykinase, succinyl-CoA synthetase, and methylmalonyl-CoA mutase, provided that if aspartokinase is deregulated as gene (i), at least a second gene (i) other than aspartokinase is deregulated; and cultivating the microorganism.

11. The process of claim 10, wherein compound A is β-lysine.

12. The process of claim 11, wherein the microorganism belongs to the genus Corynebacterium.

13. The process of claim 11, wherein the microorganism is Corynebacterium glutamicum.

14. The process of claim 11, wherein the deregulated lysine-2,3-aminomutase gene encodes a lysine-2,3-aminomutase heterologous to the microorganism.

15. The process of claim 11, wherein the recombinant microorganism comprises a lysine-2,3-aminomutase gene from Clostridium, Bacillus or Escherichia.

16. The process of claim 11, wherein the lysine-2,3-aminomutase comprises a polypeptide sequence of Clostridium subterminale, Bacillus subtilis or Escherichia coli lysine-2,3-aminomutase or a polypeptide sequence with a lysine 2,3-aminomutase activity which is at least 80% identical to the corresponding original polypeptide.

17. The process of claim 10, wherein Compound A is β-amino-ε-caprolactam.

18. The process of claim 10, wherein Compound A is ε-caprolactam.

19. The process of claim 10, wherein Compound A is ε-aminocaproic acid.
